(19) United States
(12) Patent Application Publication
(10) Pub. No.: US 2005/0256389 A1
(43) Pub. Date: Nov. 17, 2005

(54) Calculation method, calculation program and calculation system for information supporting arthroplasty

(76) Inventors: Yoshio Koga, Niigata-shi (JP); Kazuhiro Terajima, Niigata-shi (JP); Masaharu Kurita, Tokyo (JP); Kiyoei Akutsu, Tokyo (JP); Mitsuru Hamano, Tokyo (JP); Hiroaki Ogura, Tokyo (JP)

Correspondence Address: OSTROLENK FABER GERB & SOFFEN, 1180 AVENUE OF THE AMERICAS, NEW YORK, NY, 100368403, US

(21) Appl. No.: 10/495,650

(22) PCT Filed: May 17, 2002
(86) PCT No.: PCT/JP02/04790

§ 371 Date: May 17, 2005

(30) Foreign Application Priority Data
Nov. 16, 2001 (JP) 2001-352446

(51) Int. Cl. A61B 5/05; A61B 5/103; A61B 5/117

(52) U.S. Cl. 600/407; 600/587

(57) ABSTRACT

The present invention provides an arthroplasty supporting information calculation method for supporting diagnoses of a patient with knee disordered and total joint replacement to a total knee component, and an arthroplasty supporting information calculation program, and an arthroplasty supporting information calculation system. In an arthroplasty supporting terminal of the present invention, photographed X-ray images are acquired using X-ray irradiation mechanisms and a dedicated cassette base, Approximate three-dimensional data of a patient's bones is then created by transforming a display image of three-dimensional data of CT of the patient and/or sample bones to match the X-ray image in which the patient's bones are photographed. Three-dimensional coordinate values are then determined for this approximate three-dimensional data of the patient's bones. From these three-dimensional coordinate values, parameters are determined for evaluating positional relationships between at least two bones. The position of the display image of the three-dimensional data of a total knee component is then adjusted to match the display image of the approximate three-dimensional data of the patient's bones based on the three-dimensional coordinate values. The position of the total knee component is then calculated as anatomical coordinate values that represent the positions of at least two bones.

### BACKGROUND OF T INVENTION

1. Field of the Invention

The present invention relates to an arthroplasty supporting information calculation method for calculating information that supports diagnoses of a patient with knee disordered and total joint replacement, and to an arthroplasty supporting information calculation program and arthroplasty supporting information calculation system.

2. Description of Related Art

Conventionally, in a diagnosis of knee joint ailment, the patient is placed in a standing position and X-ray photography is performed from two directions (i.e., from the front and side) on the lower limbs (centering on the knee joint and including the femur, the tibia, and the fibula). A doctor then makes a diagnosis by looking at these X-ray photographs.

In the case of a patient with a severe knee joint disorders, a total joint replacement is performed. In this case, it is necessary to select a total knee component that has a configuration and size that matches the knee joint of the patient and to determine the position of the total knee component. Currently, the total knee component is selected and the position thereof is determined by superimposing X-ray photographs of knee joint taken from two directions at an equivalent magnification on a sheet on which are printed projected configurations taken from two directions of the total knee component.

However, in the above described conventional diagnosis method, because the alignment of the lower limbs (i.e., the positional relationship between the femur and the tibia) of the patient which is intrinsically three-dimensional is determined based on X-ray photographs taken from two dimensions which are two-dimensional images, a great deal of reliance is often placed on the experience and intuition of the doctor and it is easy for discrepancies to occur in the diagnosis.

Moreover, in a total knee component as well, when selecting the total knee component having a configuration and size to match the knee joint of the patient and when determining the position thereof, because three-dimensional confirmation is not possible a great deal of reliance is often placed on the experience and intuition of the doctor. Furthermore, it is difficult to accurately reproduce the position of the total knee component in an operation.

The present invention was conceived in view of the above circumstances, and is an object thereof to provide an arthroplasty supporting information calculation process for calculating information that supports diagnoses of the joint, selections of total knee component, and determinations of positions, which have hitherto depended a great deal on the experience of a doctor, and to an arthroplasty supporting information calculation program and arthroplasty supporting information calculation system.

### SUMMARY OF THE INVENTION

In order to solve the above problems, the arthroplasty supporting information calculation method according to the first aspect of the present invention is an arthroplasty supporting information calculation method for calculating information that supports a diagnosis of a patient with knee disordered and total joint replacement to a total knee component, comprising position coordinates acquisition processing (for example, step S1 to S15 of the embodiment) in which a configuration of a display image of three-dimensional data of a sample bone is transformed so as to match an X-ray image obtained by photographing bones of the patient so as to create three-dimensional data that is approximate to the bones of the patient (for example, the three-dimensional data that approximates the bones of the patient in the embodiment described below—hereinafter abbreviated to “approximate three-dimensional data”), and, in addition, in which three-dimensional coordinate values in real space are determined for the approximate three-dimensional data; and evaluation parameter calculation processing (for example, step S16 of the embodiment) in which positional relationships of at least two bones are evaluated from the approximate three-dimensional data and from the three-dimensional coordinate values.

In this arthroplasty supporting information calculation method, in the position coordinates acquisition processing, it is possible to easily form bones, whose characteristics are different in each individual patient, into a model using approximate three-dimensional data obtained by transforming a configuration of a display image of three-dimensional data of sample bones so as to match the bones of the patient. Furthermore, in the evaluation parameter calculation processing, using three-dimensional coordinate values that are obtained from this approximate three-dimensional data, it is possible for each individual to accurately determine evaluation parameters that are used to evaluate positional relationships in bones that require the total knee component.

Accordingly, diagnoses of joints of patients and operations to replace the total knee component, which have conventionally relied a great deal on the experience and intuition of the doctor, can be performed based not on determinations made using two-dimensional images, but on specific evaluation parameters determined from three-dimensional coordinate values. As a result, accurate diagnosis results or operation results that do not rely on the experience and intuition of the doctor can be obtained.

The arthroplasty supporting information calculation method according to the second aspect of the present invention is an arthroplasty supporting information calculation method for calculating information that supports a diagnosis of a patient with knee disordered and total joint replacement to a total knee component, comprising; position coordinates acquisition processing (for example, step S1 to S15 of the embodiment) in which a configuration of a display image of three-dimensional data of a sample bone is transformed so as to match an X-ray image obtained by photographing bones of the patient so as to create the approximate three-dimensional data (for example, the three-dimensional data that approximates the bones of the patient in the embodiment), and, in addition, in which three-dimensional coordinate values in real space are determined for the approximate three-dimensional data; and total knee component position calculation processing (for example, steps S17 to S19 of the embodiments) in which a configuration and size of the total knee component is selected for the display image of the approximate three-dimensional data, and, as a result of a position of the display image of the three-dimensional data of the total knee component being adjusted, a position for the total knee component is calculated as coordinate values of anatomical coordinates of a bone where the total knee component is to be placed.

In this arthroplasty supporting information calculation method, in the position coordinates acquisition processing, bones, whose characteristics are different in each individual patient, are easily formed into a model using approximate three-dimensional data obtained by transforming a configuration of a display image of three-dimensional data of sample bones so as to match the bones of the patient, and three-dimensional coordinate values obtained from this approximate three-dimensional data are calculated. Furthermore, in the total knee component position calculation processing, by adjusting the position of a display image of the three-dimensional data of the total knee component so that it matches a display image of the approximate three-dimensional data based on the three-dimensional coordinate values, it is possible to calculate the position of the total knee component in anatomical coordinates that are set from these three-dimensional coordinate values.

Accordingly, it is possible to acquire the position of the total knee component as specific anatomical coordinate numerical values. As a result, it is possible to solve conventional problems such as a that fact that great deal depends on the experience of the doctor, and the fact that it is difficult to accurately use or reproduce in the arthroplasty when the position of the total knee component once this has been decided, and it is possible to increase stability in the result of the arthroplasty, improve safety, and improve reproducibility.

In the arthroplasty supporting information calculation method according to the third aspect of the present invention, in the above described arthroplasty supporting information calculation method, the position coordinates acquisition processing derives a projection matrix that represents a projection relationship between real space and two-dimensional planes of projection coming from two directions from three-dimensional coordinate values of a group of steel balls (for example, the steel balls 6b of the embodiment) whose three-dimensional coordinate values in real space are known and two-dimensional coordinate values of the group of steel balls that appears in X-ray images that are obtained by photographing the bones of the patient from two directions, and, using this projection matrix, determines three-dimensional coordinate values of the approximate three-dimensional data.

As a result, in the position coordinates acquisition processing, the bones of a patient are recognized three-dimensionally using three-dimensional coordinate values in real space of the group of steel balls and two-dimensional coordinate values taken from two directions of the group of steel balls, and the three-dimensional coordinate values of the bones of the patient can be accurately determined. Accordingly, provided they have X-ray images taken from two directions in which appear a group of steel balls whose three-dimensional coordinate values are known, and by creating approximate three-dimensional data by transforming the configuration of the display image of three-dimensional data of sample bones so that it matches X-ray images in which bones of the patient are photographed, all of the doctors that are involved in an operation can easily acquire bone position information as accurate three-dimensional coordinate values.

In the arthroplasty supporting information calculation method according to the fourth aspect of the present invention, in the arthroplasty supporting information calculation method according to the third aspect, the arthroplasty supporting information calculation method includes reference point acquisition processing in which points that show characteristics of bones that can be observed from both of two directions are plotted on two-dimensional images that are taken from the two directions in which are displayed bones of the patient or bones that approximate a bone configuration of the patient, and reference points that show characteristics and structures of the bones are determined from the plotted points that show the characteristics of the bones, and the position coordinate acquisition processing determines three-dimensional coordinate values of the approximate three-dimensional data by calculating three-dimensional coordinate values in real space from the reference points using the projection matrix.

In this arthroplasty supporting information calculation method, using the bone reference point acquisition processing, the positions of bones, whose configuration is different for each individual patient, are extracted as position information of reference points of bones that have been formed into models using points that show the characteristics thereof. As a result, in the position coordinate acquisition processing, without using position information on all portions of the bones, it is possible to easily determine three-dimensional coordinate values of bones of the patient using a small amount of calculation processing from a projection matrix derived using position information on the reference points and X-ray images photographed from two directions.

Accordingly, even if the bones have a complex configuration, provided that the characteristic points of the bones can be determined, all of the doctors involved in the arthroplasty axe able to acquire position information on the bones as three dimensional coordinate values in a short time.

In the arthroplasty supporting information calculation method according to the fifth aspect of the present invention, in the arthroplasty supporting information calculation method according to the fourth aspect, the reference point acquisition processing determines the reference points by approximating a surface configuration of a bone from plotted points that show characteristics of the bone.

In this arthroplasty supporting information calculation method, by determining reference points after replacing a configuration that shows plotted bone characteristics with a simple diagram, it is possible to perform the bone reference point acquisition processing with an even smaller amount of calculation processing without any reduction in accuracy. Accordingly, during the drafting of an operation plan, it is possible to allocate time with priority given to other tasks (i.e., to tasks such as determining three-dimensional coordinate values of the approximate three-dimensional data, and calculating a position of the total knee component based on the determined three-dimensional coordinate values).

In the arthroplasty supporting information calculation method according to the sixth aspect of the present invention, in the arthroplasty supporting information calculation method according to the first aspect, there is included reference point acquisition processing in which points that show characteristics of bones that can be observed from both of two directions are plotted on two-dimensional images that are taken from the two directions in which are displayed bones of the patient or bones that approximate a configuration of bones of the patient, and reference points that show structures of the bones are determined as a result of a surface configuration of the bones being approximated from the plotted points that show the characteristics of the bones, and the evaluation parameter calculation processing determines the parameters using these reference points.

As a result, in the evaluation parameter calculation processing, without using three-dimensional coordinate values of all portions of the bones, it is possible to easily calculate evaluation parameters by a small amount of calculation processing from the three-dimensional coordinate values of reference points determined in the reference point acquisition processing. As a result, an effect corresponding to that of the arthroplasty supporting information calculation method according to the fourth aspect of the present invention can be obtained.

The arthroplasty supporting information calculation program according to the seventh aspect of the present invention is an arthroplasty supporting information calculation program for calculating information that supports a diagnosis of a patient with knee disordered and total joint replacement to a total knee component, that executes on a computer: position coordinates acquisition processing in which a configuration of a display image of three-dimensional data of a sample bone is transformed so as to match an X-ray image obtained by photographing bones of the patient so as to create the approximate three-dimensional data, and, in addition, in which three-dimensional coordinate values in real space are determined for the approximate three-dimensional data; and evaluation parameter calculation processing in which positional relationships of at least two bones are evaluated from the approximate three-dimensional data and from the three-dimensional coordinate values of the bones of the patient.

The arthroplasty supporting information calculation program according to the eighth aspect of the present invention is an arthroplasty supporting information calculation program for calculating information that supports a diagnosis of a patient with knee disordered and total joint replacement to a total knee component, that executes on a computer: position coordinates acquisition processing in which a configuration of a display image of three-dimensional data of a sample bone is transformed so as to match an X-ray image obtained by photographing bones of the patient so as to create the approximate three-dimensional data, and, in addition, in which three-dimensional coordinate values in real space are determined for the approximate three-dimensional data; and total knee component position calculation processing in which a configuration and size of the total knee component is selected for the display image of the approximate three-dimensional data, and, as a result of a position of the display image of the three-dimensional data of the total knee component being adjusted, a position for the total knee component is calculated as coordinate values of anatomical coordinates of a bone where the total knee component is to be placed.

The arthroplasty supporting information calculation system according to the ninth aspect of the present invention is an arthroplasty supporting information calculation system for calculating information that supports a diagnosis of a patient with knee disordered and total joint replacement to a total knee component, comprising: an X-ray image photographing device (for example, the dedicated cassette base 4 and the X-ray irradiation mechanisms 5a and 5b) that photographs an X-ray image of the patient; a position coordinates acquisition device (for example, the steel ball grouping (i.e., the frame markers) position detection section 12, the camera calibration processing section 13, the patient bone reference point detection section 14, the patient bone three-dimensional coordinates acquisition section 15, the sample bone reference point detection section 18, the sample bone three-dimensional coordinates acquisition section 19, and the three-dimensional data transformation processing section 20 of the embodiment) that transforms a configuration of a display image of three-dimensional data of a sample bone such that it matches an X-ray image obtained by photographing bones of the patient so as to create the approximate three-dimensional data, and, in addition, that determines three-dimensional coordinate values in real space for this approximate three-dimensional data; and an evaluation parameter calculation device (for example, the three-dimensional lower limb alignment calculation section 21 of the embodiment) that determines parameters for evaluating positional relationships of at least two bones from the approximate three-dimensional data and from the three-dimensional coordinate values of the bones of the patient.

The arthroplasty supporting information calculation system according to the tenth aspect of the present invention is an arthroplasty supporting information calculation system for calculating information that supports a diagnosis of a patient with knee disordered and total joint replacement to a total knee component, comprising: an X-ray image photographing device that photographs an X-ray image of the patient; a position coordinates acquisition device (for example, the steel ball grouping (i.e., the frame markers) position detection section 12, the camera calibration processing section 13, the patient bone reference point detection section 14, the patient bone three-dimensional coordinates acquisition section 15, the sample bone reference point detection section 18, the sample bone three-dimensional coordinates acquisition section 19, and the three-dimensional data transformation processing section 20 of the embodiment) that transforms a configuration of a display image of three-dimensional data of a sample bone such that it matches an X-ray image obtained by photographing bones of the patient so as to create the approximate three-dimensional data, and, in addition, that determines three-dimensional coordinate values in real space for the approximate three-dimensional data; and a total knee component position calculation device (for example, the total knee component three-dimensional data positioning processing section 23 and the total knee component position coordinates calculation section 25 of the embodiment) that selects a configuration and size of the total knee component for the display image of the approximate three-dimensional data, and, by adjusting a position of the display image of the three-dimensional data of the total knee component, calculates a position for the total knee component as coordinate values of anatomical coordinates of a bone where the total knee component is to be placed.

In the arthroplasty supporting information calculation system according to the eleventh aspect of the present invention, in the arthroplasty supporting information calculation system according to the tenth aspect, the X-ray image photographing device photographs X-ray images of bones of the patient from two directions together with a group of steel balls (for example, the steel balls 6b of the embodiment) whose respective three-dimensional coordinate values in real space are already known.

As a result, in the position coordinates acquisition device, the bones of the patient are recognized three-dimensionally using three-dimensional coordinate values in real space of the group of steel balls and two-dimensional coordinate values taken from two directions of the group of steel balls, and the three-dimensional coordinate values of the bones of the patient can be accurately determined.

The dedicated cassette base according to the twelfth aspect of the present invention is a dedicated cassette base that is used in an arthroplasty supporting information calculation system for calculating information that supports a diagnosis of a patient with knee disordered and total joint replacement to a total knee component, and that is provided with: a panel (for example, the panel 6 of the embodiment) that is held in a direction perpendicular to the bottom surface with one side of the panel that is in a direction perpendicular to the bottom surface being attached to a central shaft (for example, the central shaft 6c of the embodiment), so that the panel is able to turn from a first position to a second position around the central shaft while a patient is maintained in a standing position, and with a recording medium (for example, the imaging plates (IP) 6a of the embodiment) for photographing an X-ray image being provided on two sides of the panel.

As a result, it is possible, without moving the patient that is placed on the cassette base, to rapidly perform X-ray photography from the two directions that correspond to the first position and the second position.

In the dedicated cassette base according to the thirteenth aspect of the present invention, in the dedicated cassette base according to the twelfth aspect of the present invention, at the first position an X-ray image from a frontal direction of a patient is recorded on the recording medium on one surface of the panel, and at the second position an X-ray image from a direction other than the frontal direction of the patient is recorded on the recording medium on another surface of the panel.

As a result, the labor of replacing the recording medium during X-ray photography can be omitted and the X-ray photography can be achieved in a short time.

In the dedicated cassette base according to the fourteenth aspect of the present invention, in the dedicated cassette base according to the thirteenth aspect of the present invention, the panel is provided with a group of steel balls (for example, the steel balls 6b of the embodiment) whose three-dimensional coordinate values in real space are known.

As a result, it is possible to photograph in an X-ray image a group of steel balls that serve as a reference for recognizing the bones of the living being three-dimensionally together with the bones of the living being.

### BRIEF DESCRIPTION THE DRAWINGS

FIG. 1 is a block diagram showing the structure of an arthroplasty supporting information calculation system of an embodiment of the present invention.

FIG. 2 is a block diagram showing the structure of an arthroplasty supporting terminal.

FIG. 3 is a view showing the relationship between a group of steel balls in a three-dimensional space and an image in which these steel balls are projected onto two-dimensional coordinates.

FIG. 4A is a view showing details of a dedicated cassette base.

FIG. 4B is a view showing details of a dedicated cassette base.

FIG. 5A is a view showing a frame marker of a panel of the dedicated cassette base.

FIG. 5B is a view showing a frame marker of a panel of the dedicated cassette base.

FIG. 6 is a flowchart showing a processing sequence of the arthroplasty supporting information calculation system.

FIG. 7A is a view showing an example of the display of a CR image of a patient.

FIG. 7B is a view showing an example of the display of a CR image of a patient.

FIG. 8 is a view showing a portion of a bone for plotting reference points of the bone.

FIG. 9 is a view showing a portion of a bone for plotting reference points of the bone.

FIG. 10 is a view showing a portion of a bone for plotting reference points of the bone.

FIG. 11A is a view for showing anatomical coordinates representing a portion of a bone.

FIG. 11B is a view for showing anatomical coordinates representing a portion of a bone.

FIG. 12A is a view showing a method of determining a cortical bone point and a diaphysis central point.

FIG. 12B is a view showing a method of determining a cortical bone point and a diaphysis central point.

FIG. 13 is a view in which three-dimensional data of a sample bone and total knee component are displayed three-dimensionally.

FIG. 14 is a view in which a lower limb alignment in approximate three-dimensional data is shown three-dimensionally by balls and cylinders.

FIG. 15A is a view in which a display image of approximate three-dimensional data of bone and a display image of three-dimensional data of total knee component art superimposed and displayed as a CR image of a patients bone.

FIG. 15B is a view in which a display image of approximate three-dimensional data of bone and a display image of three-dimensional data of total knee component are superimposed and displayed as a CR image of a patients bone.

FIG. 16A is a view in which a display image of approximate three-dimensional data of bone and a display image of three-dimensional data of total knee component are superimposed and displayed as a CR image of a patients bone.

FIG. 16B is a view in which a display image of approximate three-dimensional data of bone and a display image of three-dimensional data of total knee component are superimposed and displayed as a CR image of a patients bone.

### DETAILED DESCRIPTION OF THE INVENTION

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as limited by the foregoing description and is only limited by the scope of the appended claims.

A description will now be given of the arthroplasty supporting information calculation process and the arthroplasty supporting information calculation system of an embodiment of the present invention with reference made to the drawings. In the present embodiment, as an example, a description is given of when information that supports a diagnosis of a patient with knee disordered and total joint replacement to a total knee component is calculated.

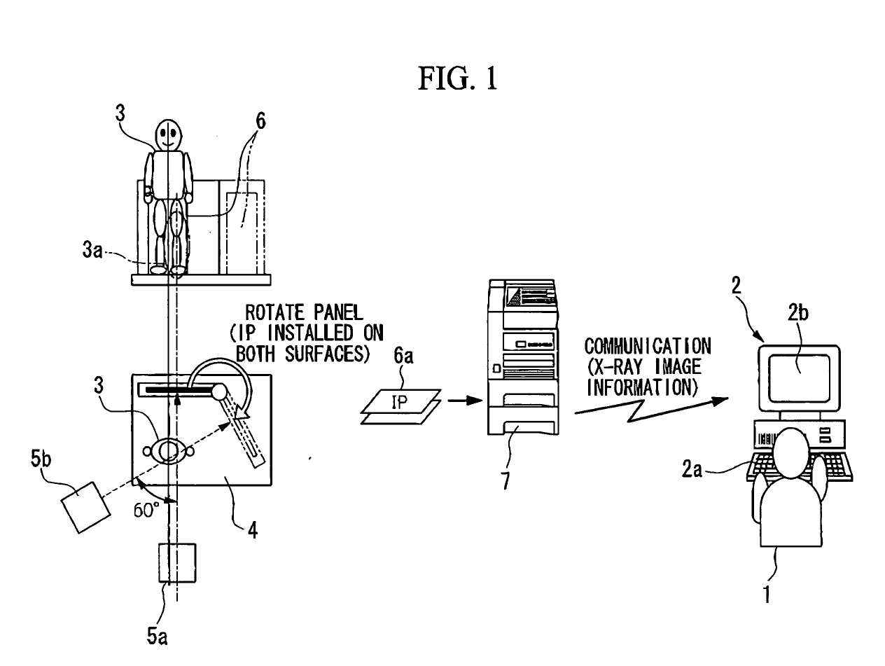

The arthroplasty supporting information calculation system of the present embodiment is shown in FIG. 1. This system is a system for realizing an arthroplasty supporting information calculation method, and is provided with an arthroplasty supporting terminal 2 that is used for calculating information required for the arthroplasty and is operated by an operator 1 (i.e., a doctor), a dedicated cassette base 4 that is used for photographing an X-ray image of a patient 3, an X-ray irradiation mechanism 5a that is used for irradiating X-rays onto the patient from a frontal direction, and an X-ray irradiation mechanism 5b that is used for irradiating X-rays onto the patient 3 from an angle of certain degrees (ex. 60 degrees). The patient 3 who is positioned on the dedicated cassette base 4 stands facing in a predetermined direction. When X-rays are irradiated towards the lower limbs 3a of the patient 3 that are to be operated on from the front and from an angle of 60 degrees using the X-ray irradiation mechanisms 5a and 5b, X-ray images of the patient 3 are recorded on a plurality of imaging plates (IP) 6a that are mounted on a panel 6. Using the image plates (IP) 6a as recording media, X-ray image information is input into the arthroplasty supporting terminal 2 via a digital X-ray system IP reader 7 and is displayed on the arthroplasty supporting terminal 2 as a computed radiographic (CR) image.

Note that it is also possible to input X-ray image information directly into the arthroplasty supporting terminal 2 from an X-ray sensor other than the imaging plates (IP) 6a.

Furthermore, the X-ray irradiation mechanisms 5a and 5b may be combined in a single X-ray irradiation mechanism, provided that this mechanism is able to move rapidly for a short time such as that in which the standing posture of the patient does not change.

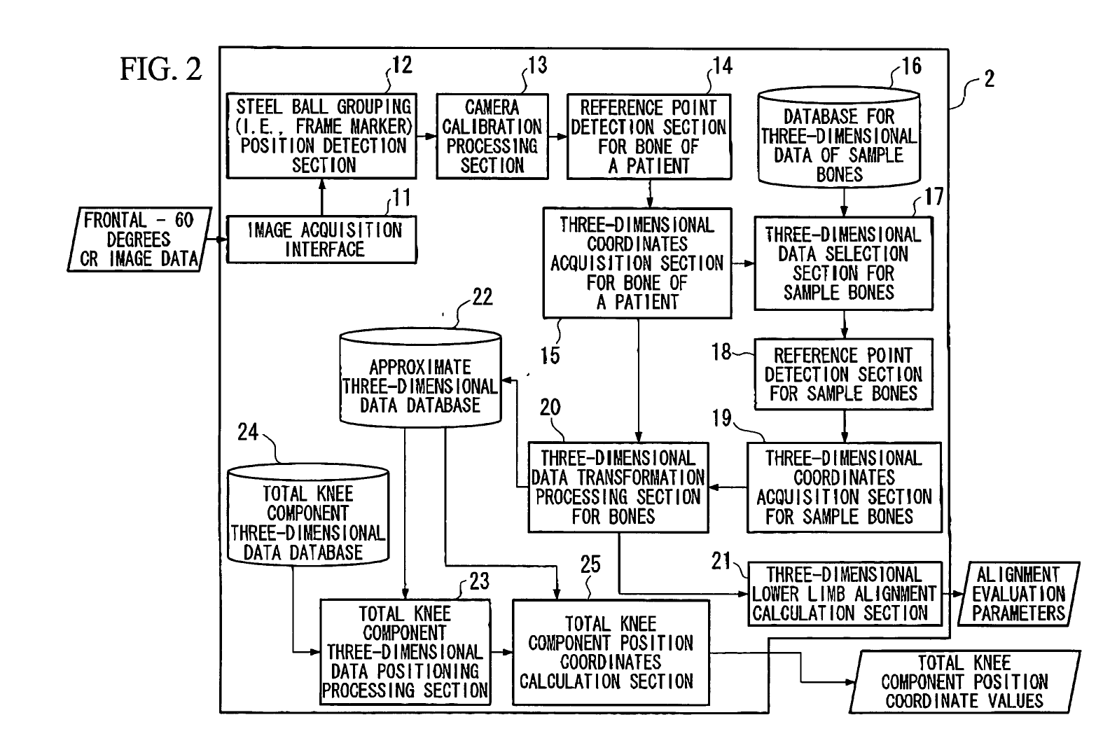

Next, using the drawings, a description will be given of the arthroplasty supporting terminal 2 that is used in the arthroplasty supporting information calculation system of the present embodiment. FIG. 2 is a block diagram that describes the functional structure of the arthroplasty supporting terminal 2. In FIG. 2, the arthroplasty supporting terminal 2 is provided with an image acquisition interface 11, a steel ball grouping (i.e., frame marker) position detection section 12, a camera calibration processing section 13, reference point detection section 14 for bones of patients, a three-dimensional coordinates acquisition section 15 for bones of patients, a database 16 for three-dimensional data of sample bones, a three-dimensional data selection section 17 for sample bones, a reference point detection section 18 for sample bones, a three-dimensional coordinates acquisition section 19 for sample bones, a three-dimensional data transformation processing section 20 for bones, and a three-dimensional lower limb alignment calculation section 21 that are used for calculating a three-dimensional lower limb alignment. Here, the term “three-dimensional data of the sample bone” refers to three-dimensional data that represents the configuration of a standard bone of patient and that is created in advance artificially.

Furthermore, the arthroplasty supporting terminal 2 is provided with an approximate three-dimensional data database 22, a total knee component three-dimensional data positioning processing section 23, a total knee component three-dimensional data database 24, and a total knee component position coordinates calculation section 25 that are used for calculating the position of a total knee component.

The image acquisition interface 11 is an interface that uses communication to acquire X-ray image information in the form of a CR image from the digital X-ray system IP reader 7.

In the steel ball grouping (i.e., frame marker) position detection section 12, in order to form a CR image for each direction by connecting together a plurality of image data, the operator 1 plots five points in an arbitrary sequence from among all the reference cross points (described below in detail) on a CR image, and obtains two-dimensional coordinate values of the reference cross points. Furthermore, the operator 1 plots all the frame marker steel balls 6b in the respective CR images from the front direction and from a direction at an angle of 60 degrees to front direction, and obtains two-dimensional coordinate values of the steel balls 6b.

The camera calibration section 13 formulates a projection equation for each of the front direction CR image and the 60 degree CR image from two-dimensional coordinate values of each of the front direction image and the 60 degree direction image of the steel balls 6b and from three-dimensional coordinate values of the steel balls 6b in real space, and calculates a projection matrix by solving this equation. Note that English alphabetic characters representing the matrices and vectors described below, including the character P showing the projection matrix, are changed for the bold face characters used in each of the equations.

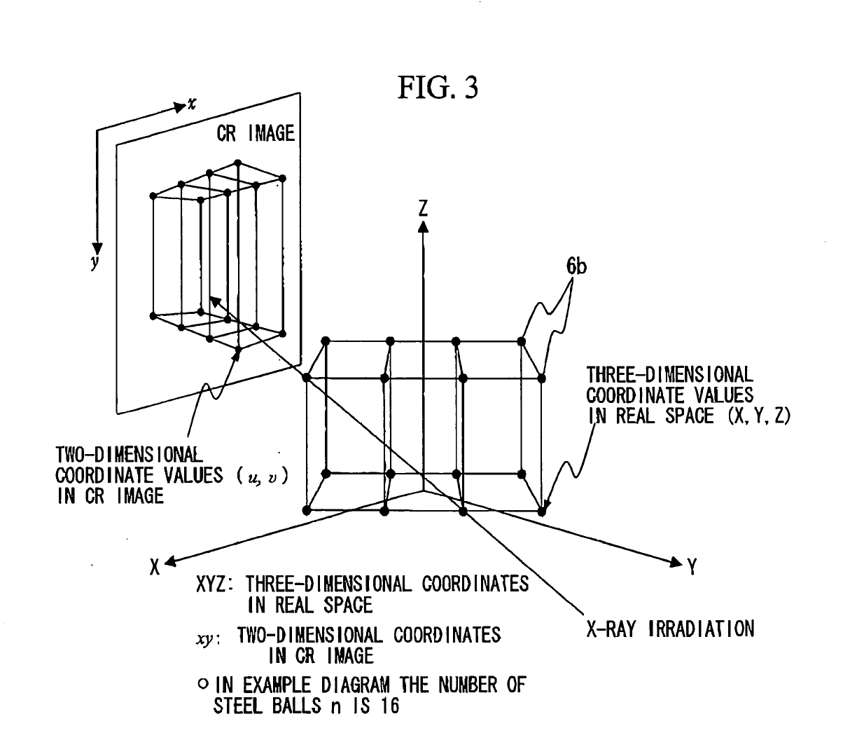

Specifically, if an explanation is given using FIG. 3, then if (u, v) are taken as the two-dimensional coordinate values in a CR image of the steel balls 6b, and if (X, Y, Z) are taken as the three-dimensional coordinate values in real space of the steel balls 6b, the projection equation is expressed as
s\tilde{m}=P\tilde{M}=A[R,t]\tilde{M} (1)
wherein
s: scalar (numerical)
\tilde{m}: expansion vector on image plane
P: projection matrix
\tilde{M}: expansion vector in three-dimensional space
A: camera internal matrix
R: rotation matrix
T: translation vector
Here, the projection matrix P is

$$P = [ \begin{matrix}{p}_{11} & {p}_{12} & {p}_{13} & {p}_{14} \\ {p}_{21} & {p}_{22} & {p}_{23} & {p}_{24} \\ {p}_{31} & {p}_{32} & {p}_{33} & {p}_{34}\end{matrix} ] = [ \begin{matrix}{p}_{1}^{T} & {p}_{14} \\ {p}_{2}^{T} & {p}_{24} \\ {p}_{3}^{T} & {p}_{34}\end{matrix} ] \qquad ( 2 )$$

Accordingly, if Formula (1) is expanded, it is possible to formulate two linear equations relating to the elements of the projection matrix P from one three-dimensional point and a two-dimensional image thereof.

$$\{ \begin{matrix}{p}_{1}^{T} {M}_{i} - {u}_{i} {p}_{3}^{T} {M}_{i} + {p}_{14} - {u}_{i} {p}_{34} = 0 \\ {p}_{2}^{T} {M}_{i} - {v}_{i} {p}_{3}^{T} {M}_{i} + {p}_{24} - {v}_{i} {p}_{34} = 0\end{matrix} \qquad ( 3 )$$

Here, if the number of steel balls 6b is n, the following equation is obtained
Bp=0 (4)
Note that the following formula shows the elements of the projection matrix P arranged in a line.
p=[p₁^{T},p₁₄,p₂^{T},p₂₄,p₃^{T}p₃₄]^{T} (5)
While the following formula is a 2n×12 matrix defined from the two-dimensional coordinate values and the three-dimensional point of the point B in Formula (3).

$$B = [ \begin{matrix}{X}_{1} & {Y}_{1} & {Z}_{1} & 1 & 0 & 0 & 0 & 0 & - {u}_{1} {X}_{1} & - {u}_{1} {Y}_{1} & - {u}_{1} {Z}_{1} & - {u}_{1} \\ 0 & 0 & 0 & 0 & {X}_{1} & {Y}_{1} & {Z}_{1} & 1 & - {v}_{1} {X}_{1} & - {v}_{1} {Y}_{1} & - {v}_{1} {Z}_{1} & - {v}_{1} \\ ⋮ & ⋮ & ⋮ & ⋮ & ⋮ & ⋮ & ⋮ & ⋮ & ⋮ & ⋮ & ⋮ & ⋮ \\ {X}_{n} & {Y}_{n} & {Z}_{n} & 1 & 0 & 0 & 0 & 0 & - {u}_{n} {X}_{n} & - {u}_{n} {Y}_{n} & - {u}_{n} {Z}_{n} & - {u}_{n} \\ 0 & 0 & 0 & 0 & {X}_{n} & {Y}_{n} & {Z}_{n} & 1 & - {v}_{n} {X}_{n} & - {v}_{n} {Y}_{n} & - {v}_{n} {Z}_{n} & - {v}_{n}\end{matrix} ] \qquad ( 6 )$$

The camera calibration processing section 13 calculates the projection matrix P by solving Formula (4) using an inverse iteration method or Newton's method or the like.

Moreover, in the patient bone reference point detection section 14, in order to determine three-dimensional coordinate values of characteristic portions of bones from CR images of the patient 3, the operator 1 plots characteristic portions (i.e., reference points) of a bone in respective CR images from a front direction and a 60 degree direction, and stores the two-dimensional coordinate values thereof in the patient bone reference point detection section.

The patient bone three-dimensional coordinates acquisition section 15 calculates three-dimensional coordinate values in real space of reference points of bones of the patient 3 using the projection matrix P calculated by the camera calibration section 13 from the two-dimensional coordinate values of the characteristic portions (i.e., the reference points) of the bones of the patient 3. Then, based on these three-dimensional coordinate values, sets anatomical coordinates for representing the potions of at least two bones.

The sample bone three-dimensional data database 16 is a database that stores three-dimensional data of sample bones. In addition, in the sample bone three-dimensional data selection section 17, in order to calculate a three-dimensional lower limb alignment, the operator 1 selects three-dimensional data of at least two sample bones from the sample bone three-dimensional database 16.

In the sample bone reference point detection section 18, in the same way as in the patient bone reference point detection section 14, in order to determine three-dimensional coordinate values of characteristic portions of sample bones, the operator 1 plots characteristic portions (i.e., reference points) of three-dimensional data of the sample bones in respective CR images from a front direction and a 60 degree direction, and stores the two-dimensional coordinate values thereof.

In the same way as in the patient bone three-dimensional coordinate acquisition section 15, the sample bone three-dimensional coordinates acquisition section 19 calculates three-dimensional coordinate values in real space of reference points of sample bones using the projection matrix P calculated by the camera calibration section 13 from the two-dimensional coordinate values of the characteristic portions (i.e., the reference points) of the three-dimensional data of the sample bones. Then, based on these three-dimensional coordinate values, sets anatomical coordinates for representing the potions of at least two bones.

The bone three-dimensional data transformation processing section 20 displays a CR image of the bones of the patient 3 superimposed with a display image of the three-dimensional data of its Ca data and/or three dimensional data of the sample bones, and determines the relationship between the three dimensional coordinate values in real space of the reference points of the bones of the patient 3 that are determined by the patient bone three-dimensional coordinates acquisition section 15 and the three-dimensional coordinate values in real space of the reference points three-dimensional data of the sample bones that are determined by the sample bone three-dimensional acquisition section 19. The bone three-dimensional data transformation processing section 20 also automatically transforms the configuration and moves the position of the display image of the three-dimensional data of the sample bones such that the three-dimensional coordinate values of the reference points in the three-dimensional data of the sample bones match the reference points of the bones of the patient 3. In addition, the bone three-dimensional data transformation processing section 20 stores approximate three-dimensional data of the patient 3 obtained by transforming the three-dimensional data of the sample bones such that it matches the reference points of the bones of the patient 3 in the approximate three-dimensional data database 22.

The three-dimensional lower limb alignment calculation section 21 calculates a three-dimensional lower limb alignment from the approximate three-dimensional data of the patient 3.

The total knee component three-dimensional data positioning processing section 23 displays a list of optional total knee components in order to calculate the position of the total knee component. The operator 1 selects the desired total knee component three-dimensional data from the total knee component three-dimensional data database 24 in which is stored total knee component three-dimensional data. Next, the total knee component three-dimensional data positioning processing section 23 displays a display image of the approximate three-dimensional data that is stored in the approximate three-dimensional data database 22 superimposed with a display image of the total knee component three-dimensional data. The operator 1 then matches the position of the display image with the position where the total knee component is actually to be placed in the arthroplasty by moving and rotating the display image of the total knee component three-dimensional data, so that the size of the total knee component and the target position in the arthroplasty are decided.

The total knee component position coordinates calculation section 25 calculates the target position in the arthroplasty that has been decided by the total knee component three dimensional data positioning processing section 23 as a target position in anatomical coordinates defied using approximate three-dimensional data that is stored in the approximate three-dimensional data database 22. Note that the target position calculated here is the position (i.e., the translation movement) and the attitude (i.e., the rotation) of the total knee component relative to the anatomical coordinates.

The sample bone three-dimensional data database 16, the approximate three-dimensional data database 22, and the total knee component three-dimensional data database 24 may be formed, for example, so as to include a recording medium that can be read from or written to by a computer such as nonvolatile memory such as a hard disk device, a magneto optical disk device, flash memory or the like, volatile memory such as random access memory (RAM), or a combination of these.

Furthermore, the image acquisition interface 11, the steel ball grouping (i.e., the frame marker) position detection section 12, the camera calibration processing section 13, the patient bone reference point detection section 14, the patient bone three-dimensional coordinates acquisition section 15, the sample bone three-dimensional data selection section 17, the sample bone reference point detection section 18, the sample bone three-dimensional coordinates acquisition section 19, the bone three-dimensional data transformation processing section 20, the three-dimensional lower limb alignment calculation section 21, the total knee component three-dimensional data positioning processing section 23, and the total knee component position coordinates calculation section 25 may each be realized by dedicated hardware. Alternatively, the functions thereof may be realized by loading a program that is formed by memory or a central processing unit (CPU) in memory and then executing this program.

An input apparatus 2a and a display apparatus 2b and the like are connected to the arthroplasty supporting terminal 2. Here, input apparatus 2a refers to an input device such as a keyboard or mouse, while display apparatus 2b refers to an image display apparatus such as a cathode ray tube (CRT) display device or a liquid crystal display device and to an aural display apparatus such as a speaker.

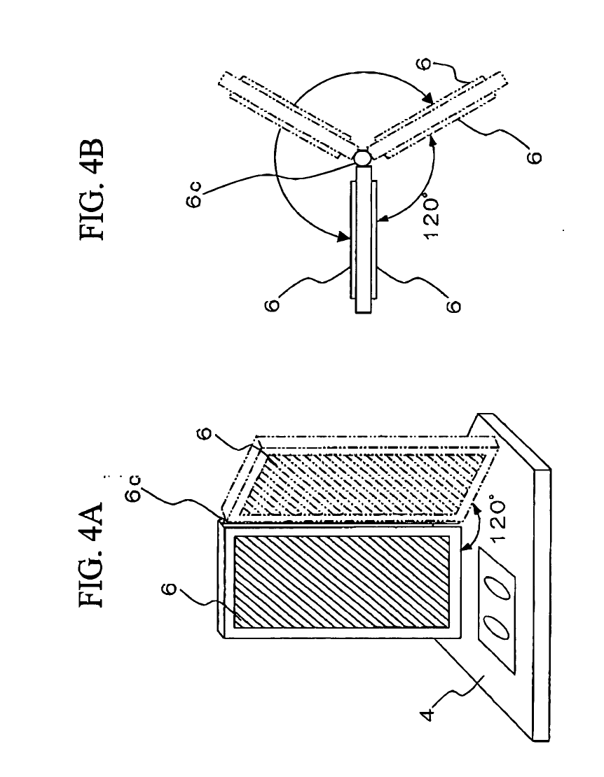

Next, a description will be given using the drawings of the dedicated cassette base 4 used in the arthroplasty supporting information calculation system of the present embodiment. FIG. 4A and FIG. 4B are views for describing in further detail the dedicated cassette base 4. In FIG. 4A, the dedicated cassette base 4 is provided with a panel 6 that, when the patient 3 is in a standing position, acquires X-ray images from two directions of the patient 3 in the same attitude. One side in the vertical direction of the panel 6 is attached to a central shaft 6c that is vertically mounted on the bottom surface of the dedicated cassette base 4. As a result, the panel 6 is able to turn without causing the patient 3 to move. An imaging plate (IP) 6a, which is a recording medium for photographing X-ray images, is provided on both an A surface and a B surface.

As is shown in FIG. 4b, by rotating the one panel 6 for 240 degrees (=360 degrees −120 degrees), the panel 6 is separated into an A surface that records a frontal X-ray image of the patient 3 corresponding to the X-ray irradiation mechanism 5a, and a B surface that records an X-ray image from a direction of 60 degrees of the patient 3 corresponding to the X-ray irradiation mechanism 5b.

Note that, if an X-ray image of a left lower limb of the patient is to be acquired an X-ray image in the frontal direction is recorded by the A surface, while an X-ray image from a direction of 60 degrees is recorded by the B surface. If an X-ray image of a right lower limb is to be acquired, this is reversed.

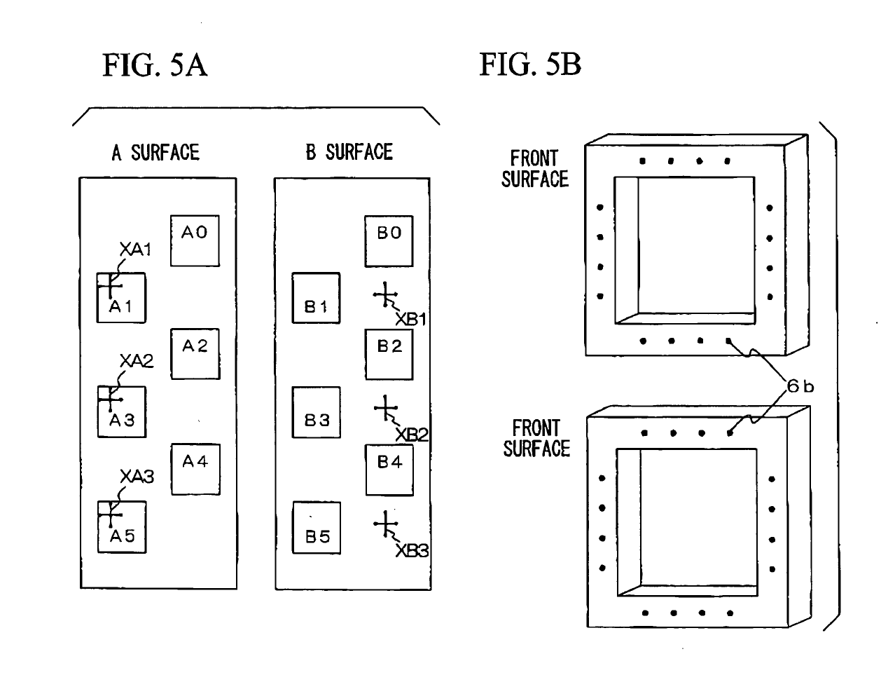

FIG. 5A, and FIG. 5B are views for describing frame markers embedded in the panel 6 of the dedicated cassette base 4. Frame markers are references used to calculate predicted positions of X-ray irradiation points. As is shown in FIG. 5A, there may, for example, be six, shown by A0 to A5, in the A surface of the panel 6, and six, shown by a B0 to B5, in the B surface of the panel 6. As is shown in FIG. 5B, steel balls 6b are embedded three-dimensionally as reference points in the front surface and rear surface of each frame marker.

Furthermore, five steel balls that form reference cross points, which become reference points of each image, are embedded in the A surface and B surface of the panel 6 so that a plurality of X-ray images can be connected so as to form a single display image.

Note that the three-dimensional coordinate values in real space of the steel balls 6b that are embedded in the frame markers, the positions in real space of the five steel balls that form the reference cross points, the distances between steel balls, and the distances between each reference cross point are all known.

Next, using the drawings, a description will be given of the processing flow of the arthroplasty supporting information calculation system of the present embodiment using as an example a case in which information is calculated that supports an operation to replace a patient knee joint with a total knee component.

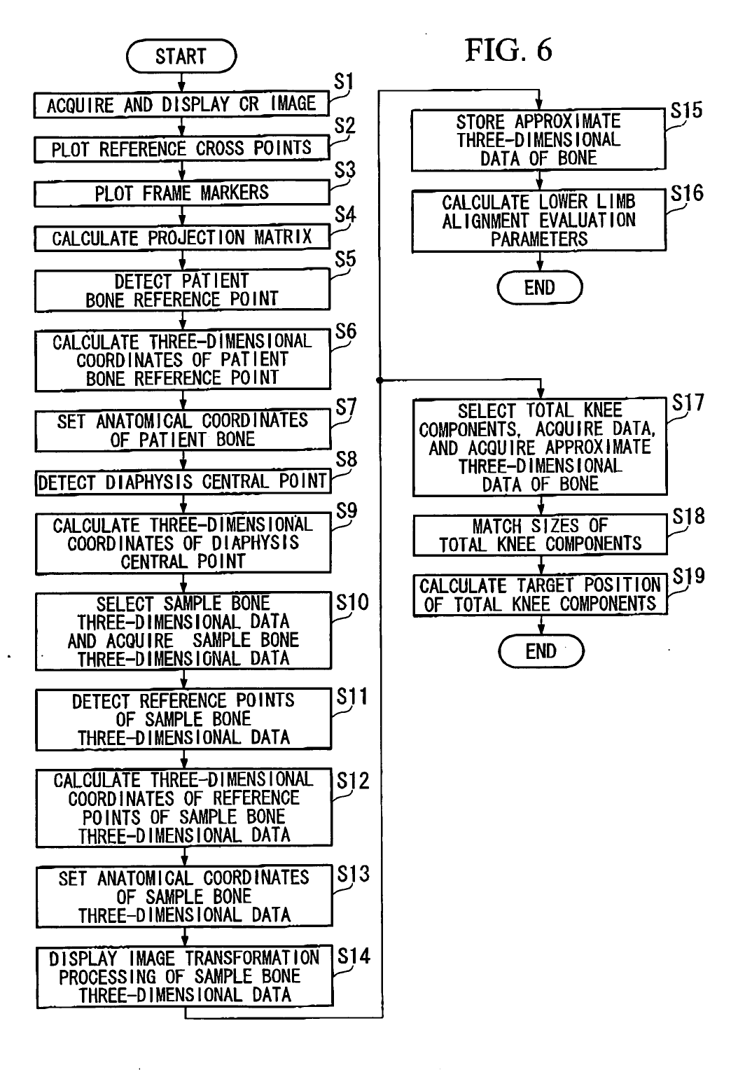

FIG. 6 is a flowchart describing the processing flow of an arthroplasty supporting information calculation system. When the operator 1 performs the arthroplasty to read CR image data from both a frontal direction and 60 degree direction of a subject patient 3 from the digital X-ray system IP reader 7, the arthroplasty supporting terminal 2 acquires the CR image data via the image acquisition interface 11 and displays it on a screen of the display apparatus 2b (step S1).

It is also possible to read CR image data directly from an X-ray sensor and display it on the screen of the display apparatus 2b without going through the digital X-ray system IP reader 7.

Next, when the operator 1 plots all of the reference cross points on the CR image, the steel ball grouping (i.e., the frame markers) position detection section 12 stores two-dimensional coordinate values of all of the plotted reference cross points. Then, based on these two-dimensional coordinate values, positional relationships of the reference cross points are automatically determined and are stored in a predetermined order (step S2).

When the plotting of the reference cross points is completed and the operator 1 has plotted all of the frame marker steel balls 6b of the CR image, the steel ball grouping (i.e., the frame markers) position detection section 12 stores two-dimensional coordinate values of all of the plotted steel balls 6b, and determines relationships between the three-dimensional coordinate values in real space of the steel balls 6b and the two-dimensional coordinate values on the CR images in the frontal direction and the 60 degree direction (step S3).

Note that using characteristic information such as the brightness and configuration of the steel balls 6b, the steel balls 6b may also be detected from the CR images automatically.

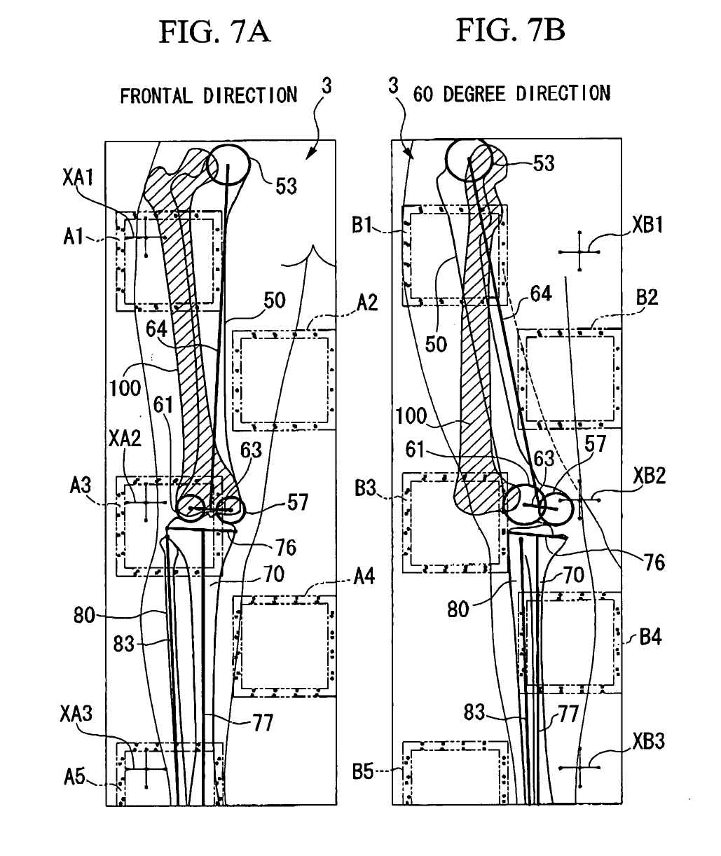

FIG. 7A and FIG. 7B are examples of displays of CR images. FIG. 7A is a CR image representing an X-ray image from the frontal direction of the patient 3, while FIG. 7B is a CR image representing an X-ray image from a 60 degree direction of the patient 3. In these drawings, with the reference cross points in the A surface of the panel 6 taken as XA1 to XA3 and the reference cross points in the B surface of the panel 6 taken as XB1 to XB3, the CR images from the respective directions are reproduced. In addition, with the frame markers in the A surface of the panel 6 taken as A1 to A5 and the frame markers in the B surface of the panel 6 taken as B1 to B5, the CR images from the respective directions are reproduced. Note that the other symbols shown and FIG. 7 are described below in detail.

Once the two-dimensional coordinate values of the CR image from the frontal direction and the CR image from the 60 degree direction of the frame marker steel balls 6b as well as the three-dimensional coordinate values in real space thereof have been determined, the camera calibration processing section 13 formulates a projection equation, as described above, from the relationships between the two-dimensional coordinate values and the three-dimensional coordinate values of the respective directions, and calculates the projection matrix P by solving this equation (step S4).

Note that if the positional relationships of the X-ray irradiation mechanisms 5a and 5b and the dedicated cassette base 4 are fixed and known, then because it is possible to calculate and store the projection matrix P in advance, step S2 to step S4 may be omitted.

Next, once the operator 1 has plotted characteristic portions (i.e., reference points) of the bones of the patient 3 in respective CR images from both a frontal direction and from a 60 degree direction in order to determine three-dimensional coordinate values of the characteristic portions of the bones from the CR images of the patient 3, the patient bone reference point detection section 14 stores all two-dimensional coordinate values of the plotted reference points (step S5).

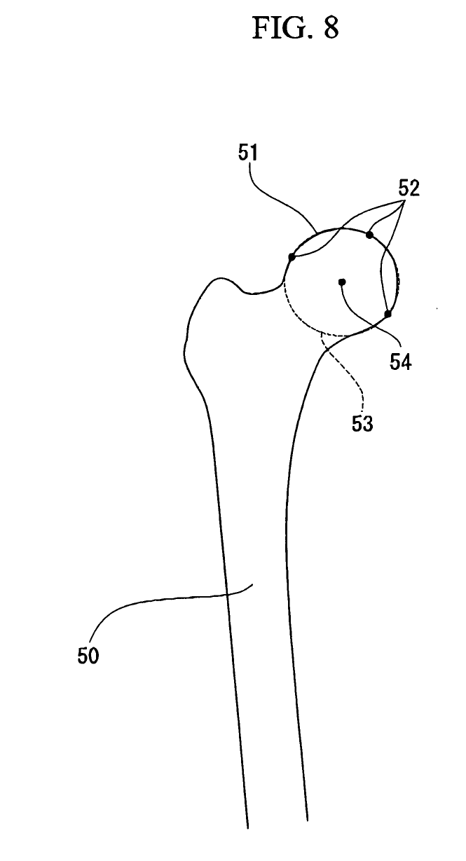

This operation will be described in detail using FIG. 8 through FIG. 10. FIG. 8 through FIG. 10 are views showing portions that are plotted as bone reference points. The respective reference points are plotted as is described below.
(1) Femoral head reference point (see FIG. 8)

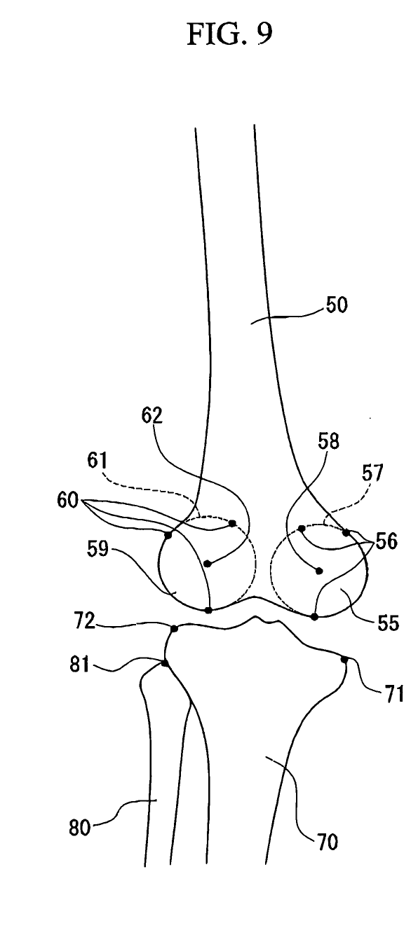

In order to determine a central point obtained by making an approximate circle of the outline of the bone head of the femur 50, the operator 1 plots three femoral head reference points 52. The patient bone reference point detection section 14 then approximates the bone head configuration as a femoral head approximate circle 53 from two-dimensional coordinate values of the (three) femoral head reference points 52, and calculates the two-dimensional coordinate values of the femoral head central point 54.
(2) Reference point of epicondylus mediale (see FIG. 9)

The operator 1 plots three reference points of medial condyle 56 in order to determine a central point that is obtained by making an approximate circle of the outline of the epicondylus mediale 55. The patient bone reference point detection section 14 then approximates the epicondylus mediale 55 as an epicondylus mediale approximate circle 57 from two-dimensional coordinate values of the (three) reference points of medial condyle 56, and calculates the two-dimensional coordinate values of a center point of medial condyle 58.
(3) Reference point of lateral condyle (see FIG. 9)

The operator 1 plots three reference points of lateral condyle 60 in order to determine a central point that is obtained by making an approximate circle of the outline of the lateral condyle 59. The patient bone reference point detection section 14 then approximates the lateral condyle 59 as an lateral condyle approximate circle 61 from two-dimensional coordinate values of the (three) reference points of lateral condyle 60, and calculates the two-dimensional coordinate values of a center point of lateral condyle 62.
(4) Tibia proximal joint surface inner edge point (see FIG. 9)

The operator 1 plots a tibia proximal joint surface inner edge point 71, and the patient bone reference point detection section 14 stores the two-dimensional coordinate values thereof.
(5) Tibia proximal joint surface outer edge point (see FIG. 9)

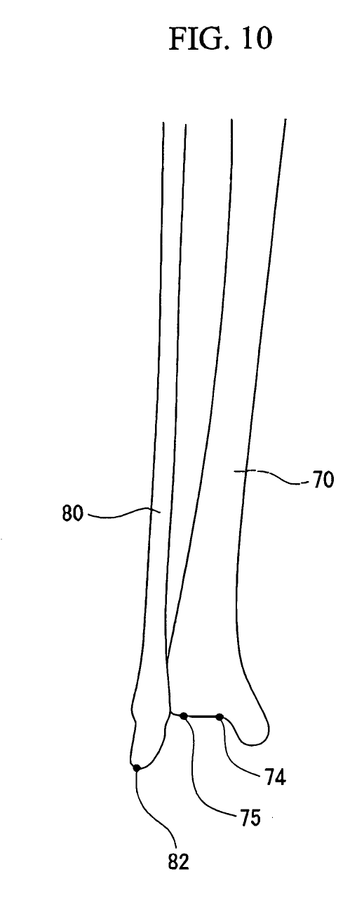

The operator 1 plots a tibia proximal joint surface outer edge point 72, and the patient bone reference point detection section 14 stores the two-dimensional coordinate values thereof.
(6) Tibia distal joint surface inner edge point (see FIG. 10)

The operator 1 plots a tibia distal joint surface inner edge point 74, and the patient bone reference point detection section 14 stores the two-dimensional coordinate values thereof.
(7) Tibia distal joint surface outer edge point (see FIG. 10)

The operator 1 plots a tibia distal joint surface outer edge point 75, and the patient bone reference point detection section 14 stores the two-dimensional coordinate values thereof.
(8) Apex of fibular head (see FIG. 9)

The operator 1 plots an apex of fibular head 81, and the patient bone reference point detection section 14 stores the two-dimensional coordinate values thereof.
(9) Distal end of fibula (see FIG. 10)

The operator 1 plots a distal end of fibula 82, and the patient bone reference point detection section 14 stores the two-dimensional coordinate values thereof.

In the patient bone reference detection section 14, characteristic reference points of the bones are plotted on the frontal direction CR image and the 60 degree direction CR image, and once the two-dimensional coordinate values thereof have been obtained, the patient bone three-dimensional coordinate acquisition section 15 calculates three-dimensional coordinate values in real space that correspond to the two-dimensional coordinate values of the reference points of the bones using the projection matrix P determined by the camera calibration processing section 13 in step S4 (step S6).

Specifically, firstly, when the three-dimensional coordinate values of the reference points are:

$$\begin{matrix}projection matrix {P}_{f} in frontal direction = [ \begin{matrix}{p}_{{f}_{11}} & {p}_{{f}_{12}} & {p}_{{f}_{13}} & {p}_{{f}_{14}} \\ {p}_{{f}_{21}} & {p}_{{f}_{22}} & {p}_{{f}_{23}} & {p}_{{f}_{24}} \\ {p}_{{f}_{31}} & {p}_{{f}_{32}} & {p}_{{f}_{33}} & {p}_{{f}_{34}}\end{matrix} ] & ( 7 \text{-} 1 ) \\ projection matrix {P}_{q} in 60 degree direction = [ \begin{matrix}{p}_{{q}_{11}} & {p}_{{q}_{12}} & {p}_{{q}_{13}} & {p}_{{q}_{14}} \\ {p}_{{q}_{21}} & {p}_{{q}_{22}} & {p}_{{q}_{23}} & {p}_{{q}_{24}} \\ {p}_{{q}_{31}} & {p}_{{q}_{32}} & {p}_{{q}_{33}} & {p}_{{q}_{34}}\end{matrix} ] & ( 7 \text{-} 2 )\end{matrix}$$

and the three-dimensional coordinate values being determined are (X, Y, Z), then if
(u_{f}, v_{f}): plotted two-dimensional coordinate values on frontal direction CR image
(u_{q}, v_{q}) plotted two-dimensional coordinate values on 60 degree direction CR image

$$\begin{matrix}B = [ \begin{matrix}{u}_{f} & {p}_{{f}_{31}} - {p}_{{f}_{11}} & {u}_{f} & {p}_{{f}_{32}} - {p}_{{f}_{12}} & {u}_{f} & {p}_{{f}_{33}} - {p}_{{f}_{13}} \\ {v}_{f} & {p}_{{f}_{31}} - {p}_{{f}_{21}} & {v}_{f} & {p}_{{f}_{32}} - {p}_{{f}_{22}} & {v}_{f} & {p}_{{f}_{33}} - {p}_{{f}_{23}} \\ {u}_{q} & {p}_{q31} - {p}_{q11} & {u}_{q} & {p}_{q32} - {p}_{q12} & {u}_{q} & {p}_{q33} - {p}_{q13} \\ {v}_{q} & {p}_{q31} - {p}_{q21} & {v}_{q} & {p}_{q32} - {p}_{q22} & {v}_{q} & {p}_{q33} - {p}_{q23}\end{matrix} ] & ( 8 \text{-} 1 ) \\ b = [ \begin{matrix}{p}_{{f}_{14}} - {u}_{f} & {p}_{{f}_{34}} \\ {p}_{{f}_{24}} - {v}_{f} & {p}_{{f}_{34}} \\ {p}_{q14} - {u}_{q} & {p}_{q34} \\ {p}_{q24} - {v}_{q} & {p}_{q34}\end{matrix} ] & ( 8 \text{-} 2 )\end{matrix}$$

then the three-dimensional coordinate values in real space are determined by:

$$( \begin{matrix}X \\ Y \\ Z\end{matrix} ) = {( {B}^{T} B )}^{- 1} \cdot {B}^{T} b \\ ( \begin{matrix}{B}^{T} \text{:} transposed matrix of matrix B \\ {( )}^{- 1} \text{:} inverse matrix of contents of brackets\end{matrix} \qquad ( 9 )$$

Next, based on the determine three-dimensional coordinate values, two anatomical coordinates are set on the femur 50 side and the tibia 70 and fibula 80 side (step S7).

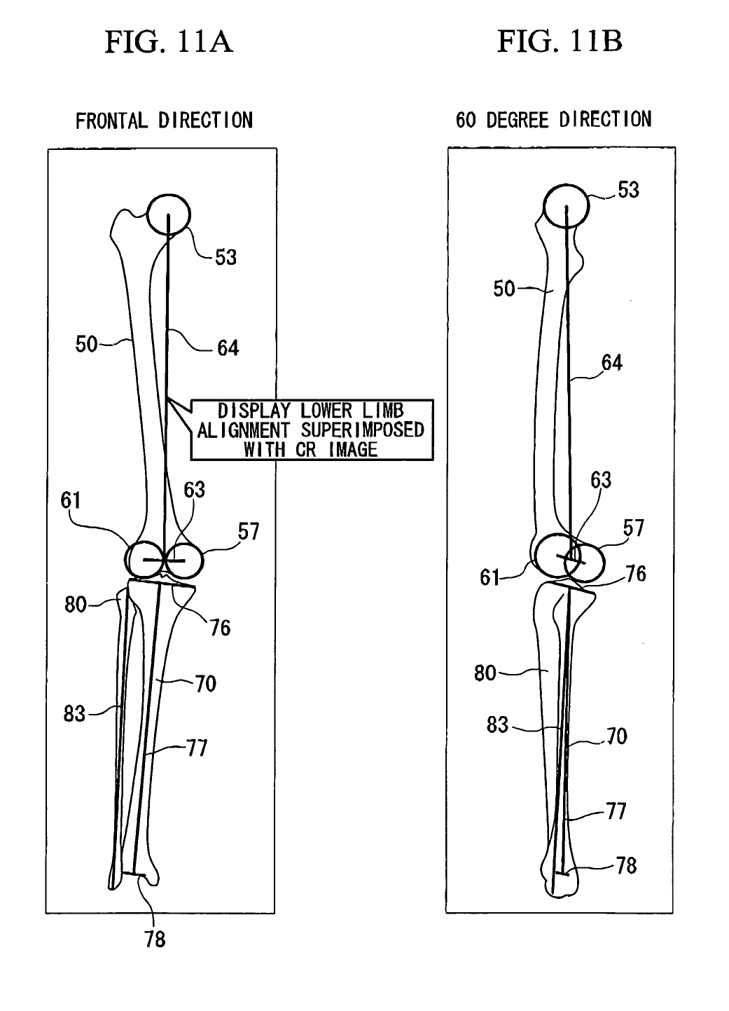

Specifically, if the two anatomical coordinates are described with reference made to FIG. 8 through FIG. 10 and to FIG. 11A and FIG. 11B that show anatomical coordinates, then they are set in the manner described below.
1. Anatomical coordinates on the femur 50 side origin; central point between the center point of medial condyle 58 and the center point of lateral condyle 62.
X axis: segment 63 connecting the center point of medial condyle 58 and the center point of lateral condyle 62.
Y axis: segment from the origin to the femoral head central point 54 that is perpendicular to X axis and segment 64 (i.e., Z′ axis).
Z axis: segment perpendicular to X axis and Y axis. The orientation of the vector determined by the vector product (i.e., the X axis×the Y axis) is positive,
2. Anatomical coordinates on the tibia 70 and fibula 80 side origin: central point between the tibia proximal joint surface inner edge point 71 and the tibia proximal joint surface outer edge point 72.
Z axis; segment 77 connecting the origin and the central point between the tibia distal joint surface inner edge point 74 and the tibia distal joint surface outer edge point 75. The orientation from the origin to the central point between the tibia distal joint surface inner edge point 74 and the tibia distal joint surface outer edge point 75 is negative.
X axis: vertical line from the Z axis that passes through the origin and that is the segment that intersects the segment 83 connecting the apex of fibular head 81 with the distal end of fibula 82. The leftward orientation heading from the X-ray irradiation mechanism is positive.
Y axis: segment perpendicular to X axis and Z axis. The orientation of the vector determined by the vector product (i.e., the Z axis×the X axis) is positive.

When the anatomical coordinates are set, because the operator 1 plots the bone cortex points on a CR image, the patient bone reference point detection section 14 calculates the two-dimensional coordinate values of the diaphysis central point (i.e., the central point between two bone cortex points) from the two-dimensional values of the bone cortex points (step S8).

Specifically, the patient bone reference point detection section 14 divides into ten segments the lengths of the femur 50, the tibia 70, and the fibula 80 (i.e., the distances from a minus end point to a plus end point on the Z axis on the anatomical coordinates of each one) using the segment that intersects with the Z axis, and projects those segments in a frontal direction and a 60 degree direction so as to display them on the screen of the display apparatus 2b. The operator 1 then plots intersection points between the bone cortex points of each bone and the ten segment dividing lines of the diaphysis sections in the frontal CR images and the 60 degree CR images. As a result, the two-dimensional coordinate values of the bone cortex points are calculated.

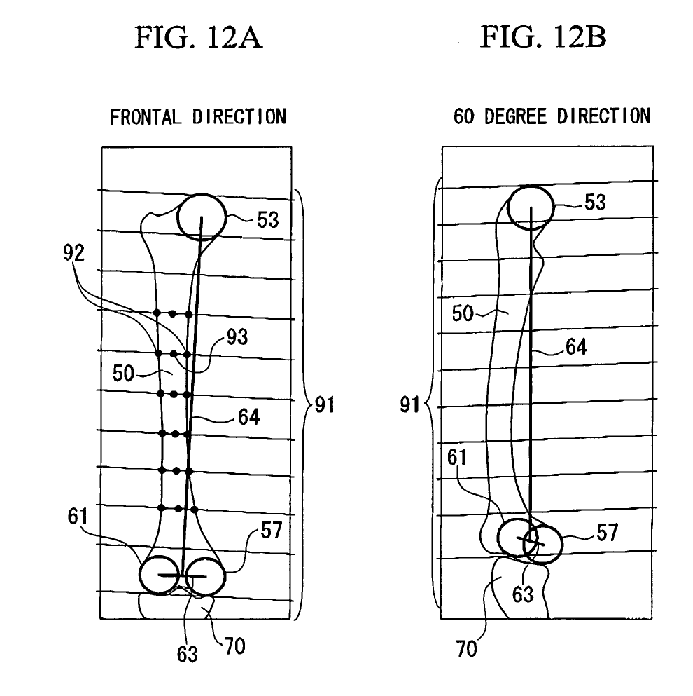

FIG. 12A and FIG. 12B are views showing a method of determining a diaphysis point and a bone cortex central point and take as the diaphysis points 92 intersection points between diaphysis points of a bone (for example, the femur 50) and the ten segment dividing lines 91 that are projected in a frontal CR image and a 60 degree CR image. The diaphysis section is the section that is sandwiched by the fourth and ninth segment dividing lines 91 (going from the top), and the diaphysis central point 93 is the central point of the two bone cortex points 92 within this section.

Once the two-dimensional coordinate values of the diaphysis central point 93 has been calculated using the patient bone reference point detection section 14, the patient bone three-dimensional coordinate acquisition section 15, in the same way as in the case of the bone reference points in step S6, calculates three-dimensional coordinate values in real space that correspond to the two-dimensional coordinate values of the diaphysis central point 93 using the projection matrix P determined by the camera calibration processing section 13 in step S4 (step S9).

The operator 1 then selects sample bone three-dimensional data of the femur 50, the tibia 70, and the fibula 80, which form the source for creating approximate three-dimensional data of the bones of the patient 3 from among lists in the sample bone three-dimensional data database 16 displayed on the display apparatus 2b. When the three-dimensional data of the sample bone is selected, the sample bone three-dimensional data selection section 17 acquires three-dimensional data of the relevant sample bone from the sample bone three-dimensional data database 16 (step S10).

When the sample bone three-dimensional data selected by the operator 1 has been acquired, the operator 1 plots reference points of the three-dimensional data of the sample bone from a frontal direction and from a 90 degree direction using the same procedure as was used in step S5. Here, the reason why the reference points are plotted from a 90 degree direction and not a 60 degree direction is in order to obtain more accurate reference coordinates. When the operator 1 has plotted the reference points, the sample bone reference point detection section 18 stores all the two-dimensional coordinate values of the plotted reference points (step S11).

Next, using the same procedure as was used in step S6, the sample bone three-dimensional coordinates acquisition section 19 calculates three-dimensional coordinate values in real space that correspond to the two-dimensional coordinate values of the reference points of the bone using the projection matrix P determined by the camera calibration processing section 13 in step S4 (step S12).

Furthermore, based on the determined three-dimensional coordinate values, using the same procedure as was used in step S7, two anatomical coordinates are set, one for the tibia and fibula side and one for the femur side of the sample bone three-dimensional data (step S13).

Note that the processing from step S11 to step S13 is performed in advance when preparing sample bone three-dimensional data and does not need to be executed each time provided that the information thereof has been recorded.

Once the reference points in the CR image of the bone of the patient 3 and the reference points of the sample bone three-dimensional data have been determined, sample bone positioning processing (step S14) is performed in the bone three-dimensional data transformation processing section 20 such that the reference points in the CR image of the bone of the patient 3 match the reference points of the sample bone three-dimensional data.

Specifically, in the first positioning processing, firstly, a relationship between reference points in the CR image of the bone of the patient 3 and reference points of the sample bone three-dimensional data is determined. Next, as was previously shown in FIG. 7A and FIG. 7B, a CR image of the bone of the patient 3 and a two-dimensional projected image of the sample bone three-dimensional data are simultaneously displayed on the display apparatus 2b. Next, the two-dimensional projected image 100 of the sample bone three-dimensional data that is displayed on the CR image of the bone of the patient 3 on the display apparatus 2b is converted using translation movement, rotation, scale conversion and the like such that three-dimensional coordinate values of corresponding reference points match each other. Here, the translation movement and rotation are performed by a general matrix operation, and the scale conversion processing is performed using a warping process or the like.

As a result of this processing, the reference points of the sample bone overlap with the reference points of the bone of the patient 3, and the position, attitude, and size of the bone is summarily determined.

Next, in the second positioning processing, as is shown in FIG. 12A and FIG. 12B, the lengths of the femur 50, tibia 70, and fibula 80 (i.e., the distances from a minus end point to a plus end point on the Z axis on the anatomical coordinates of each one) are divided into ten segments using the segment that intersects with the Z axis, and these segments are projected in a frontal direction and a 60 degree direction and are displayed on the screen of the display apparatus 2b. The operator 1 then performs movement, rotation, expansion, and contraction processing such that slice cross-sections of the sample bone at each of the ten segment dividing lines 91 match the slice cross-sections of the bone of the patient 3 in the CR image.

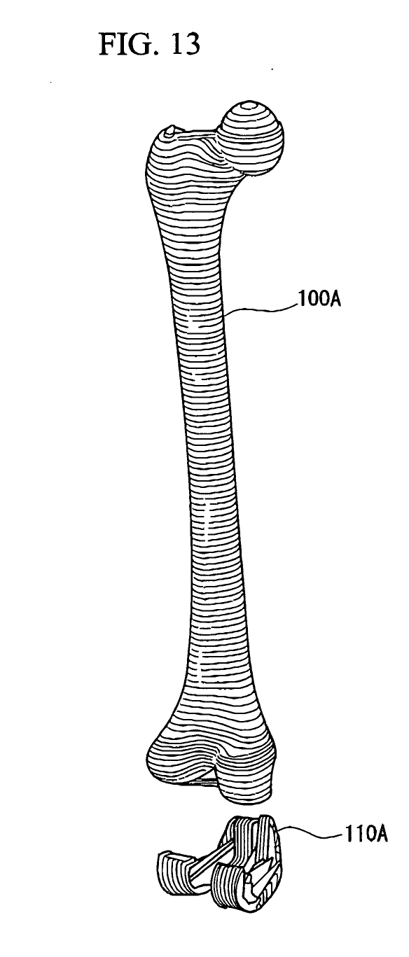

The symbol 100A in FIG. 13 is a view showing three-dimensionally the three-dimensional data of the sample bone. The bone three-dimensional data transformation processing section 20 appropriately executes movement, rotation, expansion, and contraction processing such that the slice cross-sections of the display image of the sample bone three-dimensional data that is projected and displayed on the CRP image of the bone of the patient 3 matches the outline of the bone of the patient 3 on this CR image. Note that the symbol 110A shown in FIG. 13 is described below.

Once the slice cross-sections of the display image of the sample bone three-dimensional data match the slice cross-sections of the bone of the patient 3 in the CR image at all positions of the ten segment dividing lines 91, interpolation processing is performed on the three-dimensional data between the ten segment dividing lines 91.

Note that, as a result of this processing, the sample bone matches the bone of the patient 3, and forms approximate three-dimensional data that corresponds to the patient 3.

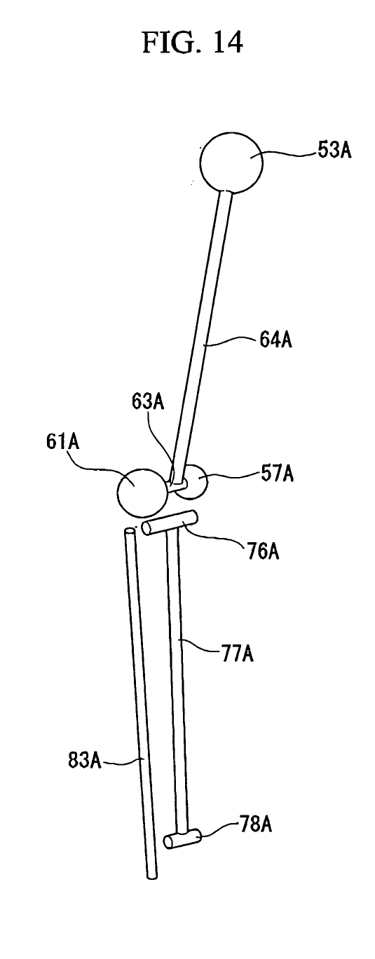

The bone three-dimensional data transformation processing section 20 stores this approximate three-dimensional data in the approximate three-dimensional data database 22 (step S15). FIG. 14 is a view in which a lower limb alignment in an approximate three-dimensional data display image is shown three-dimensionally by balls and cylinders. Here, in FIG. 14, lower limb alignments in which “A” has been added onto the symbol numbers show that they are a three-dimensional display of lower limb alignments having symbols represented by only the same symbols that are shown in FIG. 11.

Next, in order to perform a diagnosis of joint disorders of the patient 3, the three-dimensional lower limb alignment calculation section 21 calculates, for example, the 11 types of lower limb alignment evaluation parameters given below from the approximate three-dimensional data in which the aforementioned lower limb alignments are three-dimensionally displayed by balls and cylinders (step S16).

The 11 items are (1) degree of femur curvature, (2) position of maximum femur curvature, (3) degree of tibia curvature, (4) position of maximum tibia curvature, (5) femur bone angle, (6) knee joint extension angle, (7) lower limb load line transit point, (8) knee joint fissure angle, (9) femur angle of anteversion, (10) knee joint angle of torsion, and (11) tibia twist angle. The three-dimensional lower limb alignment calculation section 21 displays numerical values for the calculated three-dimensional lower limb alignments on the display apparatus 2b.

In contrast, in order to create a preplan for when the operation to replace the knee joint of the patient 3 with a total knee component is performed, the operator 1 selects total knee component of an optional size from a list that is displayed on the screen of the display apparatus 2b. The total knee component three-dimensional data positioning processing section 23 acquires approximate three-dimensional data of the bones of the patient 3 from the approximate three-dimensional data database 22, and also acquires total knee component three-dimensional data selected from the total knee component three-dimensional data database 24 (step S17).

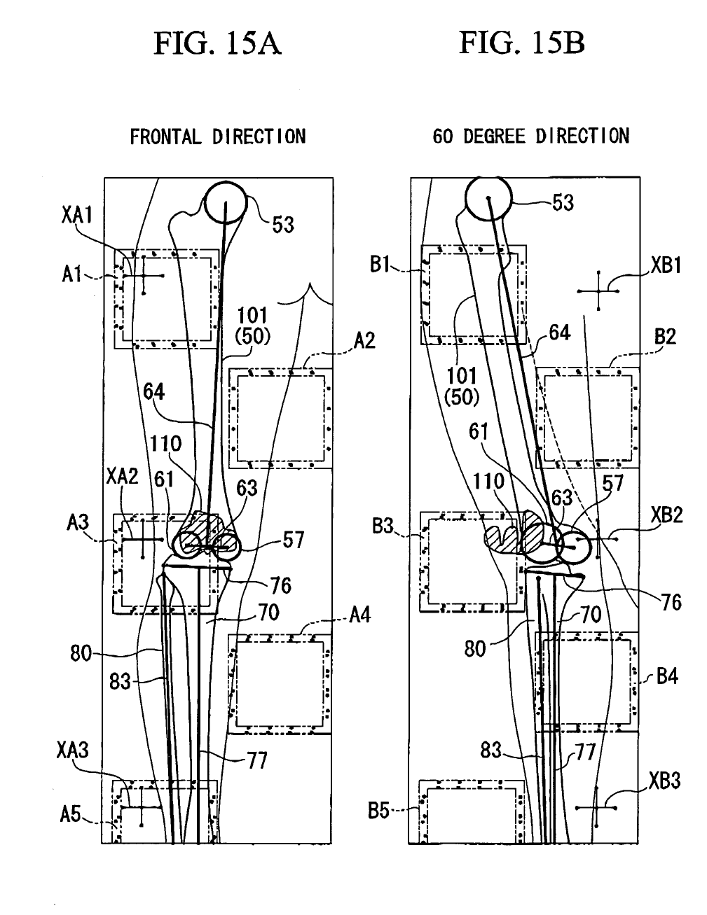

Next, as is shown in FIG. 15A and FIG. 15B, a display image 101 of the approximate three-dimensional data of the patient 3 that is stored in the approximate three-dimensional data database 22 is displayed on the display apparatus 2b simultaneously with the total knee component image 110. The operator 1 then performs translation movement and rotation on the image 110 of the total knee component that are displayed on the display image 101 of the approximate three-dimensional data of the bone on the display apparatus 2b, so as to position of the image 110 such that the total knee component match the positions where they are to be actually placed in the operation.

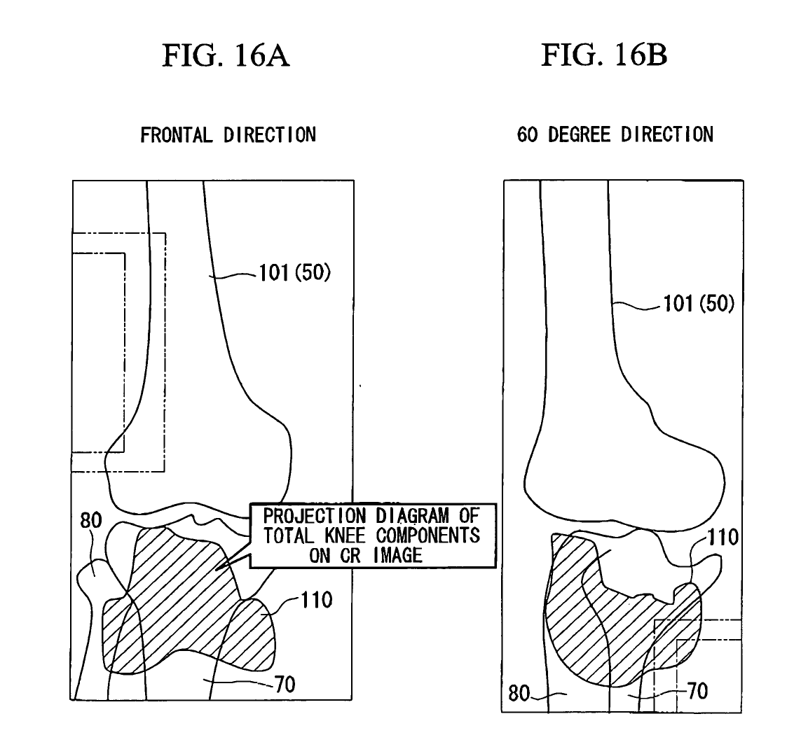

Here, if necessary, the operator 1 may repeat the selection of the three-dimensional data total knee component and the position matching thereof on the CR image until the a knee joint of a size that may be thought most suitable for the patient 3 is found. Based on the results of this, the total knee component three-dimensional data positioning processing section 23 stores the size of the components of the total knee component that is used (step S18). Furthermore, once the size of the components of the total knee component have been decided, as is shown in FIG. 16A and FIG. 16B, the operator 1 matches accurate target positions of the total knee component. Note that, in FIG. 13, which was shown previously, the image 110 of the total knee component is shown in a three-dimensional view (i.e., the symbol 110A).

When the size of the total knee component and accurate target positions have been decided by the total knee component three-dimensional data positioning processing section 23, the total knee component position coordinates calculation section 25 calculates target positions of the total knee component in anatomical coordinates of the femur and/or tibia that are defined by approximate three-dimensional data (step S19). Note that the term “target position” used here refers to the position (i.e., the translation movement) and the attitude (i.e., the rotation) of the total knee component relative to the anatomical coordinates.

Note also that, in the above described embodiment, a description is given of an example in which the arthroplasty supporting information calculation system is used when diagnosis of the patient and, in particular, the knee joint thereof, and the operation to replace this with the total knee component are performed. However, provided that it is possible to acquire X-ray images photographed from two directions, this system may be used not only for the patient and, moreover, for knee joint, but, starting with hip joint, for diagnoses of joints of any portion of all types of living bodies that have a skeleton, and for operations to replace these joints with artificial components.

Moreover, in the above described embodiment, when creating a CR image from an X-ray image of a bone of the patient 3, the arthroplasty supporting information calculation system extracts an outline of the bone (bone outline extraction processing) and emphasizes this outline (outline emphasizing processing). As a result, the CR image may be processed so as to be even more easily viewed by the operator 1. Moreover, the transformation operation is unnecessary when three-dimensional data of the bones of the actual patient are obtained by CT scan or the like.

It is also possible to perform information calculation processing that supports a diagnosis of joint disorders or an operation of the joint by recording a program that realizes the functions of the arthroplasty supporting terminal 2 in the above described embodiment on a computer readable recording medium, and then by reading and executing the program recorded on this recording medium using a computer system. Note that the term “computer system” used here includes OS and hardware such as peripheral devices. Moreover, if a WWW system is being used, the term “computer system” also includes homepage providing environments (or display environments). The term “computer readable recording medium” refers to transportable media such as flexible disks, magneto optic disks, ROM, CD-ROM and the like and to recording devices such as hard disks that are incorporated in a computer system. Furthermore, the term “computer readable recording medium” may also refer to computers that form servers and clients when the program is transmitted via a communication circuit such as a telephone line or a network such as the Internet.

The arthroplasty supporting information calculation system of the present embodiment photographs a patient 3 using X-rays from two directions using the dedicated cassette base 4, determines a projection matrix P of a CR image and of real space using frame markers whose three-dimensional coordinate values that appear on a CR image created from this X-ray image are known, and recognizes lower limbs 3a of a patient 3 that are to be operated on three-dimensionally using three-dimensional coordinate values. Then, by transforming a display image of three-dimensional data of sample bones to match the CR image of the patient 3, approximate three-dimensional data of the patient 3 is created. Evaluation parameters for evaluating positional relationships between a femur, and a tibia and fibula are then calculated from three-dimensional coordinate values determined from this approximate three-dimensional data of the bones.

Accordingly, because it is possible to photograph a patient in a standing position using the dedicated cassette base 4, the effect is obtained that it is possible to acquire an X-ray image in a state in which a load is actually applied to the knee joint due to this standing state, without placing any burden on the patient.

Moreover, by three-dimensionally recognizing the knee joint of the patient using X-ray images from two directions, in comparison with when a three-dimensional image is acquired by CT scan, the effect is obtained that the patient is only exposed to a small amount of radiation.

Furthermore, the conventional problem that the lower limb alignment (i.e., the positional relationship between the femur and tibia), which is three-dimensional, was determined using X-ray photographs from two directions, which are two-dimensional images, is solved and it is possible to three-dimensionally recognize and diagnose the bone and joint.

Moreover, as a result of the operator 1 adjusting the position of a display image of three-dimensional data of the total knee component to match a display image of approximate three-dimensional data of a bone of the patient 3 based on determined three-dimensional coordinate values, the position of a total knee component is calculated as anatomical coordinate values that represent the positions of at least to bones. Accordingly, by using information calculated using the arthroplasty supporting information calculation system of the present embodiment in a preoperative task such as selecting the total knee component and deciding the position, which have hitherto depended a great deal on the experience of the doctor, it is possible to increase stability in operation results, improve safety, and improve reproducibility in the placement of the total knee component in the target position and the like.

Furthermore, as a result of the doctor and patient simultaneously confirming information that supports the above described diagnosis and operation, which is displayed on the arthroplasty supporting terminal 2, the arthroplasty supporting information calculation system can be used as an informed consent tool for deepening the understanding and sense of security of the patient towards the diagnosis and operation.

1. An arthroplasty supporting information calculation method for calculating information that supports a diagnosis of a patient with knee disordered and total joint replacement to a total knee component, comprising:
position coordinates acquisition processing in which a configuration of a display image of three-dimensional data of a sample bone is transformed so as to match an X-ray image obtained by photographing bones of a patient so as to create three-dimensional data that is approximate to the bones of the patient, and, in addition, in which three-dimensional coordinate values in real space are determined for the three-dimensional data that is approximate to these bones of the patient; and
evaluation parameter calculation processing in which positional relationships of at least two bones are evaluated from the three-dimensional data that is approximate to the bones of the patient and from the three-dimensional coordinate values of the bones of the patient.

2. An arthroplasty supporting information calculation method for calculating information that supports a diagnosis of a diagnosis of a patient with knee disordered and total joint replacement to a total knee component, comprising:
position coordinates acquisition processing in which a configuration of a display image of three-dimensional data of a sample bone is transformed so as to match an X-ray image obtained by photographing bones of a patient so as to create three-dimensional data that is approximate to the bones of the patient, and, in addition, in which three-dimensional coordinate values in real space are determined for the three-dimensional data that is approximate to these bones of the patient; and
total knee component position calculation processing in which a configuration and size of the total knee component is selected for the display image of the three-dimensional data that is approximate to the bones of the patient, and, as a result of a position of the display image of the three-dimensional data of the total knee component being adjusted, a position for the total knee component is calculated as coordinate values of anatomical coordinates of a bone where the total knee component is to be placed.

3. The arthroplasty supporting information calculation method according to claim 1, wherein the position coordinates acquisition processing derives a projection matrix that represents a projection relationship between real space and two-dimensional planes of projection coming from two directions from three-dimensional coordinate values of a group of steel balls whose three-dimensional coordinate values in real space are known and two-dimensional coordinate values of the group of steel balls that appears in X-ray images that are obtained by photographing the bones of the patient from two directions, and, using this projection matrix, determines three-dimensional coordinate values of the approximate three-dimensional data of the bones of the patient.

4. The arthroplasty supporting information calculation method according to claim 2, wherein the position coordinates acquisition processing derives a projection matrix that represents a projection relationship between real space and two-dimensional planes of projection coming from two directions from three-dimensional coordinate values of a group of steel balls whose three-dimensional coordinate values in real space are known and two-dimensional coordinate values of the group of steel balls that appears in X-ray images that are obtained by photographing the bones of the patient from two directions, and, using this projection matrix, determines three-dimensional coordinate values of the approximate three-dimensional data of the bones of the patient.

5. The arthroplasty supporting information calculation method according to claim 3, wherein the arthroplasty supporting information calculation method includes reference point acquisition processing in which points that show characteristics of bones that can be observed from both of two directions are plotted on two-dimensional images that are taken from the two directions in which are displayed bones of the patient or bones that approximate a configuration of bones of the patient, and reference points that show characteristics and structures of the bones are determined from the plotted points that show the characteristics of the bones, and
the position coordinate acquisition processing determines three-dimensional coordinate values of the approximate three-dimensional data of the bones of the patient by calculating three dimensional coordinate values in real space from the reference points using the projection matrix.

6. The arthroplasty supporting information calculation method according to claim 4, wherein the arthroplasty supporting information calculation method includes reference point acquisition processing in which points that show characteristics of bones that can be observed from both of two directions are plotted on two-dimensional images that are taken from the two directions in which are displayed bones of the patient or bones that approximate a configuration of bones of the patient, and reference points that show characteristics and structures of the bones are determined from the plotted points that show the characteristics of the bones, and
the position coordinate acquisition processing determines three dimensional coordinate values of the approximate three-dimensional data of the bones of the patient by calculating three-dimensional coordinate values in real space from the reference points using the projection matrix.

7. The arthroplasty supporting information calculation method according to claim 5, wherein the reference point acquisition processing determines the reference points by approximating a surface configuration of a bone from plotted points that show characteristics of the bone.

8. The arthroplasty supporting information calculation method according to claim 6, wherein the reference point acquisition processing determines the reference points by approximating a surface configuration of a bone from plotted points that show characteristics of the bone.

9. The arthroplasty supporting information calculation method according to claim 1, wherein the arthroplasty supporting information calculation method includes reference point acquisition processing in which points that show characteristics of bones that can be observed from both of two directions are plotted on two-dimensional images that are taken from the two directions in which are displayed bones of the patient or bones that approximate a configuration of bones of the patient, and reference points that show structures of the bones are determined as a result of a surface configuration of the bones being approximated from the plotted points that show the characteristics of the bones, and
the evaluation parameter calculation processing determines the parameters using these reference points.

10. An arthroplasty supporting information calculation program for calculating information that supports a diagnosis of a patient with knee disordered and total joint replacement to a total knee component, that executes on a computer:
position coordinates acquisition processing in which a configuration of a display image of three-dimensional data of a sample bone is transformed so as to match an X-ray image obtained by photographing bones of a patient so as to create three-dimensional data that is approximate to the bones of the patient, and, in addition, in which three-dimensional coordinate values in real space are determined for the three-dimensional data that is approximate to these bones of the patient; and
evaluation parameter calculation processing in which positional relationships of at least two bones are evaluated from the three-dimensional data that is approximate to the bones of the patient and from the three-dimensional coordinate values of the bones of the patient.

11. An arthroplasty supporting information calculation program for calculating information that supports a diagnosis of a patient with knee disordered and total joint replacement to a total knee component, that executes on a computer:
position coordinates acquisition processing in which a configuration of a display image of three-dimensional data of a sample bone is transformed so as to match an X-ray image obtained by photographing bones of a patient so as to create three-dimensional data that is approximate to the bones of the patient, and, in addition, in which three-dimensional coordinate values in real space are determined for the three-dimensional data that is approximate to these bones of the patient; and
total knee component position calculation processing in which a configuration and size of the total knee component is selected for the display image of the three-dimensional data that is approximate to the bones of the patient, and, as a result of a position of the display image of the three-dimensional data of the total knee component being adjusted, a position for the total knee component is calculated as coordinate values of anatomical coordinates of a bone where the total knee component is to be placed.

12. An arthroplasty supporting information calculation system for calculating information that supports a diagnosis of a patient with knee disordered and total joint replacement to a total knee component, comprising:
an X-ray image photographing device that photographs an X-ray image of a patient;
a position coordinates acquisition device that transforms a configuration of a display image of three-dimensional data of a sample bone such that it matches an X-ray image obtained by photographing bones of a patient so as to create three-dimensional data that is approximate to the bones of the patient, and, in addition, that determines three-dimensional coordinate values in real space for the three-dimensional data that is approximate to these bones of the patient; and
an evaluation parameter calculation device that determines parameters for evaluating positional relationships of at least two bones from the three-dimensional data that is approximate to the bones of the patient and from the three-dimensional coordinate values of the bones of the patient.

13. An arthroplasty supporting information calculation system for calculating information that supports a diagnosis of a patient with knee disordered and total joint replacement to a total knee component, comprising:
an X-ray image photographing device that photographs an X-ray image of a patient;
a position coordinates acquisition device that transforms a configuration of a display image of three-dimensional data of a sample bone such that it matches an X-ray image obtained by photographing bones of a patient so as to create three-dimensional data that is approximate to the bones of the patient, and, in addition, that determines three-dimensional coordinate values in real space for the three-dimensional data that is approximate to these bones of the patient; and
a total knee component position calculation device that selects a configuration and size of the total knee component for the display image of the three-dimensional data that is approximate to the bones of the patient, and, by adjusting a position of the display image of the three-dimensional data of the total knee component, calculates a position for the total knee component as coordinate values of anatomical coordinates of a bone where the total knee component is to be placed.

14. The arthroplasty supporting information calculation system according to claim 12, wherein the X-ray image photographing device photographs X-ray images of bones of the patient from two directions together with a group of steel balls whose respective three-dimensional coordinate values in real space are already known.

15. The arthroplasty supporting information calculation system according to claim 13, wherein the X-ray image photographing device photographs X-ray images of bones of the patient from two directions together with a group of steel balls whose respective three-dimensional coordinate values in real space are already known.

16. A dedicated cassette base that is used in an arthroplasty supporting information calculation system for calculating information that supports a diagnosis of a patient with knee disordered and total joint replacement to a total knee component, and that is provided with:
a panel that is held in a direction perpendicular to the bottom surface with one side of the panel that is in a direction perpendicular to the bottom surface being attached to a central shaft, so that the panel is able to turn from a first position to a second position around the central shaft while a patient is maintained in a standing position, and with a recording medium for photographing an X-ray image being provided on two sides of the panel.

17. The dedicated cassette base according to claim 16, wherein at the first position an X-ray image from a frontal direction of the patient is recorded on the recording medium on one surface of the panel, and at the second position an X-ray image from a direction other than the frontal direction of the patient is recorded on the recording medium on another surface of the panel.

18. The dedicated cassette base according to claim 16, wherein the panel is provided with a group of steel balls whose three-dimensional coordinate values in real space are known.

19. The dedicated cassette base according to claim 17, wherein the panel is provided with a group of steel balls whose three-dimensional coordinate values in real space are known.
